(19) United States
(12) Patent Application Publication
(10) Pub. No.: US 2024/0010580 A1
(43) Pub. Date: Jan. 11, 2024

(54) PROCESS FOR THE ONE-STEP CONVERSION OF CARBON DIOXIDE AND RENEWABLE HYDROGEN TO LOW-CARBON METHANE

(71) Applicant: Infinium Technology, LLC, Sacramento, CA (US)

(72) Inventors: Robert Schuetzle, Sacramento, CA (US); Dennis Schuetzle, Grass Valley, CA (US); Anja Rumplecker Galloway, San Rafael, CA (US); Orion Hanbury, Sacramento, CA (US); Harold Wright, Menomonee Falls, WI (US)

(73) Assignee: Infinium Technology, LLC, Sacramento, CA (US)

(21) Appl. No.: 18/445,173

(22) Filed: May 8, 2023

Related U.S. Application Data
(60) Provisional application No. 63/473,936, filed on Jul. 8, 2022.

(51) Int. Cl. C07C 1/12; B01J 23/78; B01J 23/755; B01J 23/02; B01J 23/00; B01J 35/02; B01J 37/18

(52) U.S. Cl. CPC C07C 1/12; B01J 23/78; B01J 23/755; B01J 23/02; B01J 23/005; B01J 35/026; B01J 37/18; C07C 2523/78; C07C 2523/755

(57) ABSTRACT

The objective of the present invention is to take advantage of new and improved processes and catalysts that can facilitate the efficient, direct CO₂ conversion (CO₂C) reaction to e-methane at temperatures less than about 350° C. in one step.

### CROSS-REFERENCE TO RELATED APPLICATION

This-application claims the benefit of U.S. Provisional Patent Application No. 63/473,936, filed Jul. 8, 2022, the entire content of which is hereby incorporated by reference into this document.

### FIELD OF THE INVENTION

The field of the invention is a process to produce pipe-line quality e-methane from low carbon electricity and captured CO₂ in one step and at temperatures below about 350° C.

### BACKGROUND OF THE INVENTION

The increase in global atmospheric CO₂ concentrations has been linked to changes in the earth's climate. The combustion of fossil fuels in various engines and thermal systems produces atmospheric CO₂. Concerns about climate change have led to significant societal changes toward renewable or low carbon electricity. This has also led to increasing activity to decarbonize the economy.

Renewable or low-carbon electric power can be used to produce synthetic methane, kerosine, methanol or other chemicals, which can be gases or liquids. This process is often referred to as Power-to-X (also P2X) which refers to several electricity conversion, energy storage, and reconversion pathways that use renewable power from wind, solar, nuclear, and other sources. P2X in some cases may also refer to the use of surplus electric power, typically during periods where fluctuating renewable energy generation exceeds load.

Specifically, low-carbon methane produced using renewable electric power is referred to as “e-methane”. Electrical power produced from solar, wind or other sustainable sources is commonly called “decarbonized electricity”. This renewable energy can be used to produce low-carbon H₂ using electrolysis. The H₂ when mixed with captured CO₂ can be converted to low-carbon e-methane. One of the advantages of e-methane is that it can be introduced into the well-established natural gas pipeline distribution system; compressed and transported to another location where the e-methane is converted to H₂ for the production of low-carbon fuels and chemicals; used as a low-carbon energy source for heating and cooling; compressed and used as a low-carbon fuel for engines; and as a low-carbon alternative to many other natural gas uses (Yugo, 2019).

Currently at least two main steps are required to produce e-methane from low-carbon H₂ and captured CO₂. The first step is the hydrogenation (usually referred to as the reverse water gas shift, or RWGS, reaction) of the CO₂ to produce CO and H₂ (syngas) when excess H₂ is used in the process. The CO₂ hydrogenation is typically carried out catalytically. The second step is the catalytic conversion of the syngas to e-methane.

In order to convert the CO₂ efficiently, the CO₂ hydrogenation reaction requires temperatures of greater than about 700° C. at the inlet. The objective of the e-methane is that is that it allows for a significant reduction in emissions, since it is produced from decarbonized hydrogen and CO₂. Due to this objective, it is required that the processes use electrical heaters powered by renewable energy. This represents a large amount of renewable energy input since the CO₂ hydrogenation is endothermic.

The conversion of the syngas to CH₄ is highly exothermic—giving off a heat that needs to be removed continuously from the reactor system.

Several processes to convert syngas derived from coal or biomass were developed in the 1970's. Technology development ceased following the drop in oil price, and are now primarily used as purification steps. Currently, methanation of CO and CO₂ are widely applied in ammonia synthesis plants, as well as refineries and hydrogen plants.

Thus, the state-of-the-art requires the simultaneous provision of input heat in the syngas formation step, and removal of heat in the syngas conversion step (Aryal et al, 2021; Li et al, 2022). Although the latter partially offsets the former, the net requirement is an input of heat to facilitate the integrated process. Furthermore, and of great practical significance, the process management of these two processes present engineering and operational challenges—with resultant deleterious impacts on both process efficiency and capital cost requirements.

There is accordingly a need in the art for novel processes to produce methane.

### BRIEF SUMMARY OF THE INVENTION

The invention describes an improved, one-step process for e-methane production from renewable H₂ and CO₂ mixtures taking advantage of new and improved catalysts and processes that can perform the CO₂C reaction efficiently at temperatures less than 350° C.

### BRIEF DESCRIPTION OF THE DRAWINGS

FIG. 1 shows the process of converting CO₂ and H₂ to e-CH₄ using a CO₂C reactor. For this figure: Stream 1 is low-carbon electric power; Stream 2 is water; Stream 3 is H2; Stream 4 is O2; Stream 5 is CO2; Stream 6 is CO2C Reactor feed; Stream 7 is CO2C Product; Stream 8 is portion of CO2C product; Stream 9 is e-methane; Stream 10 is water; Stream 11 is CO2 recovered; Unit 1 is Electrolyzer; Unit 2 is CO2C Reactor; Unit 3 is Product Processing Unit.

FIG. 2 shows the CO₂C process as described in Example 1. For this figure: Unit 200 is CO2C Rx; Unit 201 is Water Knockout; Unit 202 is Amine Contactor; Unit 203 is Amine Regenerator; Unit 204 is H2 Pressure Swing Adsorption; Stream 101 is Feed to CO2C Rx; Stream 102 is Reactor Effluent; Stream 103 is Water; Stream 104 is Methane, H2, CO2; Stream 105 is Methane, H2 feed to 204; Stream 106 is CO2; Stream 107 is H2; Stream 108 is Methane; Stream 109 is CO2/Amine Solution; Stream 110 is Amine to Contactor.

FIG. 3 shows a multi-tubular CO₂C reactor as described in Example 1. For this figure: 111 is Water/Stream from Steam Drum; 112 is Saturated Steam to Steam Drum.

FIG. 4 shows the CO₂C process as described in Examples 2 and 3. For this figure: Unit 200 is CO2C Reactor; Unit 201 is Water Knockout; Unit 202 is Amine Contactor; Unit 203 is Amine Regenerator; Unit 401 is Syngas Methanation Reactor System; Unit 402 is Methanation Reactor Product Separator; Stream 301 is Feed to CO2C Rx; Stream 302 is Reactor Effluent; Stream 303 is Water; Stream 304 is Methane, H2, CO2; Stream 305 is Methane, H2 feed to 401; Stream 306 is CO2; Stream 307 is Methanation Reactor Effluent; Stream 308 is Methane; Stream 309 is CO2; Stream 310 is Methanation Produced Water; Stream 311 is CO2/Amine Solution; Stream 312 is Amine to Contactor; Stream 313 is Medium Pressure Steam.

### DETAILED DESCRIPTION OF THE INVENTION

A “direct” one-step process would have the substantial advantage of reducing the requirements of these two (coupled) thermal effects—into a single, slightly exothermic (net) reaction with a required heat management challenge of significantly reduced magnitude. The benefits derived from this reaction engineering improvement, relative to the state-of-the-art, include increased process efficiency, operational simplicity, and reduced capital equipment costs.

The objective of the present invention is to take advantage of new and improved processes and catalysts that can facilitate the efficient, direct CO₂ conversion (CO₂C) reaction to e-methane at temperatures less than about 350° C. in one step.

The catalyst described in this document for the conversion of CO₂ and H₂ at low temperatures typically meets the following specifications:

1. The catalyst contains low-cost and readily available constituents and nominal levels (<0.3%) of precious metals.

2. The catalyst is robust (e.g., Rockwell hardness greater than Mohr 03-04).

3. It is chemically and physical stable up to about 1,100° C. (2,012° F.). This means that the physical property measurements such as BET surface area are within 20%, more preferably 10%, of the ambient temperature.

4. It can be loaded readily into tubular catalytic reactors.

5. The pressure drop in the catalytic reactor is acceptable.

6. The catalyst activation (reduction with H₂) can be carried out in-situ at 275-350° C.

7. The conversion of CO₂ to CH₄ is greater than about 85% at 275-350° C.

8. The selectivity of CH₄ production is greater than about 99% at 275-350° C.

9. The catalyst has a long lifetime (2-3 years) and it doesn't require systematic re-activation (reduction).

Since the catalytic conversion of CO₂ and H₂ mixtures to CH₄ is very exothermic, this reaction should typically be carried out in a tubular reactor with an effective method of removing the heat from the tubes. The maximum, practical operating temperature for a tubular reactor is 275-325° C. (Wikipedia, 2022). Although fluidized and slurry bed reactors can operate at higher temperatures up to 325-350° C., such reactors have not been developed for distributed plants (e.g., less than about 25,000 barrels/day of product output) (Steynberg, 2004).

The most common catalysts are nickel-based since this metal is abundant and inexpensive. Table 1 summarizes the performance of several Ni-based catalysts at 275-400° C. However, the Ni-based catalysts listed in Table 1 suffer from sintering, the creation of gas-phase nickel carbonyls, and formation of carbon deposits (Li et al, 2022; Tan et al, 2022). The only catalyst that was tested for more than 40 hours was reported by Onrubia-Calvo (2022) for which the CO₂ conversion decreased by about 3% after 72 hours.

Alarcron (2019) described the performance of a 30 wt. % nickel catalyst impregnated on Al₂O₃ and the 30 wt. % Ni catalyst with a 30 wt. % CeO₂ promoter impregnated on Al₂O₃. The catalysts were activated at 500° C. resulting in a catalyst surface area of about 5 m²/g. The 30% Ni on Al₂O₃ converted a 4.0/1.0 mixture of H₂/CO₂ to CH₄ at 300° C. with a CO₂ conversion efficiency of 34% and a selectivity of 100%. The addition of 30% CeO₂ increased the CO₂ conversion efficiency to 57% with a selectivity of 100%. However, the CO₂ conversion efficiency rapidly dropped to 10% at 250° C. The catalyst activation temperature of 500° C. is not feasible to achieve in a commercial scale tubular reactor, and the CO₂ conversion efficiencies of 57% at 300° C. don't meet the required minimum CO₂ conversion efficiency of 85% at this temperature. Although, the CO₂ conversion efficiency remained constant for the Ni/CeO₂ catalyst for 110 hours, this is not a long enough test to assess long-term catalyst performance.

Jia et al (2019) described a zirconia (ZrO₂) supported Ni catalyst for CO₂ methanation which was synthesized by impregnation followed by plasma calcining at 150° C. or thermal calcining at 500° C. The catalyst was reduced with H₂ at 500° C. The efficiency of CO₂ conversion was 72% and 35% at 300° C. for the plasma and thermally calcined catalyst, respectively. Since this catalyst was tested for less than 40 hours, the long-term durability of this catalyst is not known. In addition, it would not be possible to reduce the catalyst in-situ at 500° C.

Although Ru, Pd and Rh based catalysts show much better long-term stability than the Ni-based catalysts, these precious metal catalysts are expensive, and they do not meet the requirements of better than 85% CO₂ conversion efficiency at 275-350° F.

TABLE 1
Performance of Nickel-Based Catalysts for CO₂ Methanation

| | Catalyst | H₂/CO₂ Ratio | T ° C. | % CO₂ | % CH₄ |
|---|---|---|---|---|---|
| Reference | Composition | (GHSV: Hr⁻¹) | (° F.) | Conversion | Selectivity |
| Alarcron (2019) | 30% Ni on Al₂O₃ | 4.0/1.0 | 300 (572) | 34% | 100% |
| | | (36.0K) | | | |
| Alarcron (2019) | 30% Ni/30% CeO₂ | 4.0/1.0 | 250 (482) | 10% | 100% |
| | on Al₂O₃ | (36.0K) | | | |
| Alarcron (2019) | 30% Ni/30% CeO₂ | 4.0/1.0 | 300 (572) | 57% | 100% |
| | on Al₂O₃ | (36.0K) | | | |
| Rui (2020) | 9.5% Ni on CeO₂ | 4.0/1.0 | 300 (572) | 74% | 99% |
| | | (56.0K) | | | |
| Rui (2020) | 9.5% Ni on CeO₂ | 4.0/1.0 | 275 (527) | 35% | 96% |
| | | (56.0K) | | | |
| Onrubia-Calvo | 8.5% Ni on CeO₂ | 4.0/1.0 | 300 (572) | 52-49% | 98% |
| (2022) | | (56.0K) | | (0-72 hrs.) | |
| Jia (2019) | Ni on ZrO₂ | 4.0/1.0 | 300 (572) | 72% | 99% |
| | | (55.0K) | | | |
| Jiang (2018) | Ni on Bentonite | 4.0/1.0 | 300 (572) | 85% | 100% |
| | | (3.6K) | | | |
| Lin (2018) | Ni on Al₂O₃/ ZrO₂ | 4.0/1.0 | 300 (572) | 77% | 100% |
| | | (6.6K) | | | |
| Garbarino (2015) | 20% Ni on Al₂O₃ | 5.0/1.0 | 400 (752) | 81% | 96% |
| | | (55.0K) | | | |

The improved catalysts for the efficient conversion of CO₂ and H₂ to CH₄ described in this document consist of the solid-solution catalysts Ni₂Mg, Cu₃Ni or Cu₂Mg. These solid solution catalysts are formed at high temperatures (up to 1,100° C.) when the two metals in the catalysts shown above have similar crystal structures, atomic radii, electronegativities and valences.

The Ni₂Mg catalyst was manufactured by mixing two moles of nickel acetate powder with one mole of magnesium acetate powder and producing pellets of the appropriate size (typically 3-10 mm) using a high-pressure, pellet press. The pellets were then calcined at 1,000-1,100° C. The Cu₃Ni and Cu₂Mg catalysts were produced in a similar manner by producing pellets from the metal acetate salts of Cu, Ni and Mg, and then calcining the pellets at 1,000-1,100° C. The nitrate salts of the Cu, Ni and Mg may also be used to produce the catalyst but NO₂ and NO emissions are produced during the calcining process which is difficult to control. The calcining of acetates produces primarily CO₂ with some acetic acid. The acetic acid emissions are relatively easy to control by water scrubbing.

The Ni₂Mg, Cu₃Ni and Cu₂Mg catalysts are robust, and they do not change chemically and physically up to about 1,000° C. As an example, the conversion efficiency of the Ni₂Mg catalyst did not decrease (less than 2%) over a period of 1,750 operating hours in a process development unit (PDU). After the tests were completed, about 500 mg of the catalyst from the PDU was loaded into a laboratory tubular reactor and heated to 650° C. under an air flow of 100 standard cubic centimeters per minute (sccm) to determine if any carbon (coke) was formed. The exhaust from the reactor was continuously monitored with a mass spectrometer to detect the formation of CO₂, which would be consistent with carbon burning off. Since no CO₂ was detected, it was verified that no coke was formed.

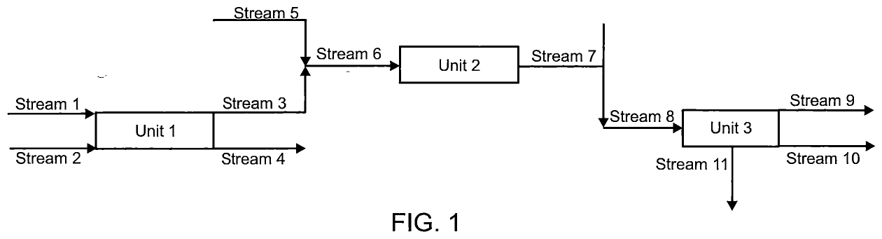

FIG. 1 shows an integrated process to produce methane from CO₂ and low-carbon electric power. Feed stream 1 is the low-carbon electric power. Low-carbon electric power includes but is not limited to wind power, solar power nuclear power, power generated from biomass or renewable natural gas, and hydropower. Feed stream 2 is water. These two feed streams are used in Unit 1, the electrolyzer. In the electrolyzer, water and low carbon energy are used to produce H₂ and O₂. H₂ is produced by electrolysis of water.

$${H}_{2} O = {H}_{2} + \frac{1}{2} {O}_{2}$$

Electrolyzers consist of an anode and a cathode separated by an electrolyte. Different electrolyzers function in slightly different ways. Different electrolyzer designs that use different electrolysis technology that are used include alkaline electrolysis, polymer electrolyte membrane (PEM) electrolysis, solid oxide electrolysis, high temperature electrolysis and other emerging types of electrolysis. Different electrolytes that are used including the liquids KOH and NaOH, and with or without activating compounds. Activating compounds are added to the electrolyte to improve the stability of the electrolyte. Most ionic activators for the hydrogen evolution reaction are composed of an ethylenediamine-based metal chloride complexes. Different electrocatalysts are used on the electrodes including many different combinations of metals and oxides like Raney-Nickel-Aluminum, which are enhanced by adding cobalt or molybdenum to the alloy.

The products from the electrolyzer are a stream comprising H₂ called stream 3 in FIG. 1 and a stream comprising O₂ called stream 4. Because renewable or low-carbon energy sources are utilized, the electrolyzer produces green H₂. Other forms of H₂ generation that may use renewable sources may also be used including pyrolysis and gasification of biomass; steam reforming of hydrocarbons with carbon capture, steam reforming of CH₄ in biogas; and other processes. H₂ may also be sourced from geological sources.

In FIG. 1, stream 5 is a stream that comprises CO₂. CO₂ is obtained from several sources. There are may stationary sources that produce large amounts of CO₂ including power plants, fertilizer production, ethanol plants, municipal sewage treatment systems, steel and iron manufacturing, oil refining, chemical production, and cement plants.

Typically, an alkylamine is used to remove the CO₂ from the gas stream. Alkylamines used in the process include monoethanolamine, diethanolamine, methyl diethanolamine, diisopropylamine, aminoethoxy ethanol, or combinations thereof. Metal Organic Framework (MOF) materials have also been used as a means of separating CO₂ from a dilute stream using chemisorption or physisorption. Other methods to produce concentrated CO₂ steams include chemical looping combustion where a circulating metal oxide material captures the CO₂ produced during combustion processes. CO₂ can also be captured from the atmosphere in what is called direct air capture (DAC).

Captured CO₂ can be converted into useful low-carbon products such as fuels (e.g., synthetic natural gas, diesel fuel, gasoline blend stocks, jet fuel, and other fuels) and chemicals (e.g., solvents, olefins, alcohols, aromatics, etc.). This is what is meant by low-carbon, very low-carbon, zero carbon, or negative carbon fuels and chemicals.

The CO₂ that comes from industrial or biological process, or is captured from the atmosphere, or that is available from a commercial CO₂ pipeline is not generally pure. These CO₂ streams typically contain sulfur containing compounds up to 2,000 parts per million and hydrocarbons up to 10 vol. %. Purification of the CO₂ including the removal of sulfur containing compounds and hydrocarbons is necessary to avoid issues with downstream processing. After purification, the purified CO₂ is suitable for the generation of low-carbon or zero-carbon fuels and chemicals as per this invention.

At least a portion of the stream 3 comprising hydrogen is blended with the stream 5 comprising CO₂ to produce a stream 6 or CO₂C Reactor feed stream. The ratio of H₂/CO₂ is from 2 to 6, or preferably from 3 to 4. The CO₂ and H₂ in stream 6 are reacted to products in a CO₂C reactor shown as Unit 2 in FIG. 1. The CO₂C reactor operates at temperatures of 250 to 425° C., and more preferably from 275 to 350° C. With a nickel-based catalyst, operating temperatures below 220° C. should be avoided due to formation of highly toxic nickel carbonyls. The primary reactions that may occur in the CO₂C reactor are the following:

CO₂+H₂=CO+H₂O(ΔH₂₉₈=+42.1 kJ/mole) Eq. 1

CO+3H₂=CH₄+H₂O(ΔH₂₉₈=−206.1 kJ/mole) Eq. 2

CO₂+4H₂=CH₄+2H₂O(ΔH₂₉₈=−165 kJ/mole) Eq. 3

The catalytic methanation of CO and CO₂ has been widely studied since its discovery by Sabatier and Senderens in 1902. The first reaction, Eq. 1, is the Reverse Water Gas Shift reaction. At standard temperature, the reaction is endothermic and in other processes requires much higher temperatures than the preferred CO₂C reactor temperatures. The second reaction, Eq. 2, is the methanation reaction where CO and H₂ are reacted to produce CH₄ and H₂O. Methanation is very exothermic as can be seen with the high negative enthalpy of reaction

The improved catalysts described previously, Ni₂Mg, Cu₃Ni or Cu₂Mg, are used in the CO₂C reactor to facilitate the chemical reactions. These catalysts may also be supported on spinels with surface areas greater than about 10 m²/g)), such as magnesium aluminate at concentrations up to about 25 wt. %.

The product from the CO₂C reactor is shown as stream 7 in FIG. 1. At least a portion of stream 7 becomes stream 8 and is fed to the Product Processing unit, Unit 3 in FIG. 1. The product processing unit can be a combination of various processes that allow the separation or additional processing of the CO₂C reactor product stream. Various embodiments are done depending on the products, yields, and selectivity observed in the CO₂C reactor. Each of the reactions that occur in the CO₂C reactor produces water. So, the first step in the product processing unit is to reduce the temperature of reactor product stream. This can be done by many different means including heat exchange with the cooler feed stream, heat exchange with other cooler stream, and with a cooling water heat exchanger. The objective of the cooling is to allow the condensation of water that may be produced in the CO₂C.

When the CO₂ conversion in the CO₂C reactor is less than about 90%, then the unreacted CO₂ is removed prior to further processing after the water removal. CO₂ removal from the stream can be done by any number of available techniques. The removal methods include the use of physical solvents. Physical solvents include refrigerated methanol that is used in the Rectisol process and the Solexol process that uses dimethyl ethers of polyethylene glycol to capture the CO₂ from the gas stream. Other means include the use of membranes selective for CO₂ removal. Another means for CO₂ removal is to use aqueous solutions of alkylamines (referred to as amines) to chemically capture the CO₂. The amine contactor captures the CO₂ in the solution. The overhead of the amine contactor is the CO₂ free gas and the amine solution with the CO₂ is heated in an amine regenerator where the CO₂ is released, and the amine aqueous solution is recycled back to the contactor. Various amines can be used such as diethanolamine (DEA), monoethanolamine (MEA), methyl-diethanolamine (MDEA), and Diisopranolamine (DIPA).

The CO₂ stream recovered from the CO₂ capture unit is shown as Stream 11 in FIG. 1. The stream is compressed and recycled and blended with the CO₂ feed stream, as shown as stream 5 in FIG. 1.

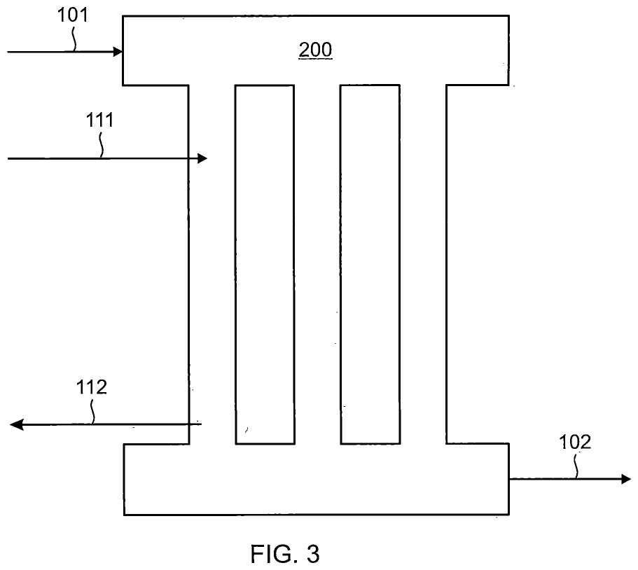

Since the conversion of CO₂ and H₂ mixtures to CH₄ is highly exothermic, the design of the CO₂C reactor is critical. Management of the heat is a key element of the reactor design. In one embodiment of the invention, the CO₂C reactor is a multi-tubular fixed bed reactor (FIG. 3). The inner reactor tubes are filled with a mixture of steam and water. The reaction heat is removed through the production of steam in the shell of the reactor. The inner diameter of the tubes is between 18 and 50 mm. The tubes are filled with catalyst particles. The catalyst particle size is optimized to minimize the pressure drop across the reactor. This small diameter of the reactor tubes means that as the feed gas reacts on the catalyst, the heat is transferred from no more than 9 to 25 mm to the heat transfer surface. Outside of the tubes, the mixture of steam and water allow heat transfer primarily through the vaporization of the water to steam. The water comes from a steam drum. Boiler Feed Water (BFW) is fed to the steam drum and circulates the system. The pressure of the steam system sets the temperature of the steam/water mixture. The pressure of the steam ranges from 200 to 1600 psig. At 1200 psig (82.7 barg), the temperature of saturated steam is 298° C. At 1100 psig (75.8 barg), the temperature of saturated steam is 292° C. At 1000 psig (69.0 barg), the temperature of saturated steam is 285° C. As such, saturated steam from 1000 to 1200 psig is an ideal cooling fluid for the CO₂C reaction that occurs at 300° C. Typically, water is fed from the steam drum to the water entrance to reactor shell and a mixture of about 50% steam and 50% water is at the steam exit of the reactor shell back to the steam drum.

In one embodiment, the CO₂C reactor is a multi-tubular reactor. The reactor tubes have an inner diameter of 18 to 50 mm and are filled with catalyst. In this embodiment, heat transfer is done with a hot oil system. Oil as a working fluid is pumped to the reactor shell. Heat is transferred from the tubes to the oil raising the temperature of the oil. The rate of oil circulation is controlled by the pumping rate that sets the heat removal rate. The hot oil leaving the reactor shell is cooled in an external heat exchanger. Cooling water, refrigerated water, or propylene glycol or other suitable materials can be used in the cold side of the external heat exchanger. The cooled hot oil is then pumped back to the reactor shell. Several suitable oils, have working temperatures that are useful for this service including Therminol XP, Therminol 55, Therminol SP, Therminol 59, or any equivalent or similar oil working fluid. These high temperature fluids allow the tubular reactor to be operated up to about 350° C.

In one embodiment the CO₂C reactor is a multi-tubular reactor is cooled with molten salt.

In one embodiment, the catalyst inside the CO₂C reactor can be diluted. Dilution of the catalyst aids in the heat transfer so that the rate of exothermic heat generation per unit volume is less and as such the local temperature rise is less. Suitable diluents include alumina, or other refractory materials.

In one embodiment, catalyst activity is modified by the amount of active metal loading in formed catalyst. The amount of active metal in the catalyst is defined to optimize the activity and thermal profile across the reactor, and should be between 1-50 weight percent.

In one embodiment of the invention, the feed gas to the CO₂C reactor can be diluted with non-reactive gases. Feed gas dilution aids in heat transfer and temperature control in several ways. It reduces the reaction heat generated per unit time per unit volume. It also adds additional mass flow rate that acts as a heat sink. Various dilution gases can be used. The ideal dilution gas is easily separable from the reactor effluent. As the reactor effluent is cooled, the condensed steam can easily be removed from the effluent by a knockout vessel or separator and since steam is a reaction product, the design change involves just increasing the size of the separator.

In one embodiment, the CO₂C reactor is a series of adiabatic reactors or reactor beds. Each reactor bed length or residence time is set such that the maximum conversion that can occur keeps the bed temperature rise to a preset temperature rise of no more than 30° C., preferably less than 20° C., and more preferably less than 15° C. Catalyst and feed gas dilution can also be used to keep the temperature rise to the preset limit. At the outlet of the reactor bed, the effluent gas is cooled in a heat exchanger that produces high pressure saturated steam in the range of 1000 to 1200 psig. This cools the reactor effluent to a temperature similar to the previous bed inlet temperature. The cooled effluent is then fed to a subsequent bed. The total number of beds is chosen to get the desired overall CO₂ conversion. Optionally, additional hydrogen or hydrogen and CO₂ can be added to the subsequent bed feed gas.

In one embodiment, metal partitions can be placed inside the CO₂C reactor tubes prior to the addition of the catalyst such that from the center of the tube to the wall of the tube there are metal pathways or connections. This aids in heat transfer since the high thermal conductivity of the metal increases the rate of heat transfer from the hot center of the tube to the cooled tube walls.

In one embodiment, the reaction is performed in a single adiabatic reactor with at 2-6 distinct reactor zones, or preferably 2-3 reactor zones that are separated by a cooling medium. The inter stage zones are externally cooled by a cooling medium.

In one embodiment, the heat transfer and heat management in the reactor are controlled using sequential H₂ injection. The initial H₂/CO₂ ratio is below stoichiometric for the net reaction. This keeps the CO₂ conversion low, and the temperature rise in the reactor bed low. H₂ at a temperature substantially below the reactor bed temperature is added at the end of the bed. The cold H₂ lowers the overall gas temperature to about the previous bed inlet temperature. The additional hydrogen with the previous bed CO₂ reacts in the subsequent bed. The number of reactor beds and cold hydrogen additions sets the overall CO₂ conversion.

In one embodiment, the CO₂C reactor is operated at low CO₂ conversion but with high overall gas recycle. This allows the temperature in the reactor bed to be controlled and the temperature rise within the acceptable range. The space velocity or residence time in the reactor bed is high such that conversion is low. Gas dilution and catalyst dilution can be used to aid in the control of the temperature.

A simpler option is to operate the catalyst at about 225° C. which converts a 4.0/1.0 H₂/CO₂ to CH₄ with a CO₂ conversion efficiency of about 40% (Table 3). In this manner, about 3 recycle loops would yield a CO₂ conversion of about 90%.

The reactor effluent is fed to the Product Processing Unit where the water in the effluent is removed by cooling and separation and the unreacted CO₂ and H₂ are removed and compressed and recycled and blended with the fresh feed to the CO₂C reactor.

The e-methane produced by the invention meets specifications for natural gas pipelines in the United States and Europe and can therefore be transported and co-mingled with natural gas. Typical specifications for interstate natural gas pipelines in the United States are shown in AGA Report No. 4 A as shown below.

Representative Specifications for Pipeline Quality Gas

| Major Components | Minimum Mol % | Maximum Mol % |
|---|---|---|
| Methane | 75 | None |
| Ethane | None | 10 |
| Propane | None | 5 |
| Butanes | None | 2 |
| Pentanes and heavier | None | 0.5 |
| Nitrogen and other inerts | None | 3 |
| Carbon dioxide | None | 2-3 |
| Total diluent gases | None | 4-5 |

Trace Components:
Hydrogen sulfide: 0.25-0.3 g/100 scf (6-7 mg/m³)
Total sulfur: 5-20 g/100 scf (115-460 mg/m³)
Water Vapor: 4.0-7.0 lb/MM scf (60-110 mg/m³)
Oxygen: 1.0%
Heating calculated (gross, saturated): 950-1,150 Btu/scf (35,400-42,800 kJ/m³)

To be clear, each pipeline can and often does set specifications more stringent than the above for pipeline quality gas. Various other nations have different pipeline quality gas practices and standards.

The e-methane product produced as per the invention, meets the representative specification for pipeline quality natural gas and can be distributed in pipelines. In one embodiment, the invention produces no C2+. In one embodiment of the invention, the overall CO₂ conversion in the CO₂C reactor is greater than about 95% and as such there is no need for CO₂ removal systems downstream of the CO₂C reactor.

### Example 1

In this example, CO₂ and H₂ are converted to CH₄ using the Ni₂Mg, Cu₃Ni or Cu₂Mg solid-solution catalyst. H₂ and CO₂ were fed to the CO₂C reactor with a molar H₂/CO₂ ratio of 4.0. Table 3 shows the conversion of CO₂ conversion to CH₄ and CO as a function of temperature from laboratory data.

As can be seen in Table 3, substantial conversion of CO₂ with high selectivity to CH₄ occurs at temperature from 250-350° C. In this example, the CO₂C reactor effluent was sent to the product separator. The amount of CO formed in the CO₂C reactor in this example was low at 2% or less. At a temperature of 300° C., the CO₂ conversion was high at 87%.

Further as an example, the catalyst was used in an integrated process to produce low carbon intensity methane at a CO₂C reactor temperature of 300° C. The CO₂ conversion was 87 mol % with 100% selectivity to CH₄.

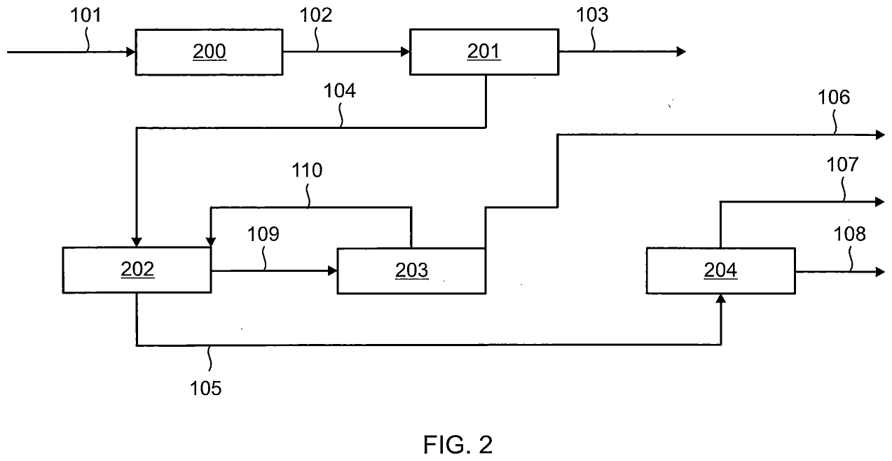

FIG. 2 shows the process configuration for this example. Hydrogen was produced from the electrolysis of water. CO₂ was captured from an industrial source and purified. The H₂ and CO₂ were mixed and shown as stream 101 in FIG. 2. The molar H₂ to CO₂ was 4 to 1. Unit 200 was the CO₂C reactor that used the above Ni₂Mg catalyst. The reactor effluent was stream 102. The stream was cooled to 40° C. and the water product was knocked out in unit 201. The water rich product stream is shown as stream 103. Stream 104 was the stream with a composition of 34 mol % H₂, 9 mol % CO₂, and 57 mol % methane that feeds unit 202. Unit 202 is an amine contactor. In this example, MDEA was used as the amine. The overhead of Unit 202 is the CO₂ free gas, stream 105, with a composition of 63 mol % CH₄ and 37 mol % H₂. The CO₂-rich amine stream, stream 109, left Unit 202 and was fed to Unit 203, the amine regenerator. The regenerated was heated through a steam reboiler that heats the amine solution and allowed the captured CO2 to be released by the amine solution and to leave the top of the amine regenerator. The captured CO₂ is shown as stream 106. The CO₂ free amine is shown as stream 110 and was pumped back to the amine contactor.

TABLE 3
Catalytic Conversion of CO₂ to CH₄ and CO as a function
of temperature for the crushed Ni₂Mg catalyst (not impregnated
on a substrate) using 5.0 cc of catalyst in 5/16″ OD Inconel Tube;
H₂/CO₂ = 4.0; 368 psi; 200 sccm)

| | % CH₄ Formed | | % CO Formed | % CO₂ |
|---|---|---|---|---|
| T ° C. | (selectivity) | | (selectivity) | Converted |
| 450 | 79 | (98) | 2 (2) | 81 |
| 400 | 83 | (100) | 0 (0) | 83 |
| 350 | 86 | (100) | 0 (0) | 86 |
| 350 | 87 | (100) | 0 (0) | 87 |
| 300 | 87 | (100) | 0 (0) | 87 |
| 275 | 85 | (100) | 0 (0) | 85 |
| 250 | 79 | (100) | 0 (0) | 79 |

Stream 105 was sent to an H₂ pressure swing adsorption (PSA) unit, shown as Unit 204. By use of pressure cycling, the H₂ was separated from the CH₄. Stream 107 is the pure H₂ product that was recycled back to the front end of the system and blended with the hydrogen in stream 3 of FIG. 1. Stream 108 is the CH₄ product stream.

The overall chemical reaction for the CO₂C reactor in this example is CO₂+4H₂=CH₄+2H₂O (g) with an enthalpy of reaction of −165 KJ/mol at 25° C. The reaction is very exothermic at the high CO₂ conversion. As such, the CO₂C reactor in this example was a multi-tubular fixed bed reactor. In this example, the saturated steam pressure produced in the shell of the reactor was 1,100 psig with a temperature of 292° C. A portion of the steam produced by the multi-tubular reactor was used as the steam to heat the amine regenerator reboiler to allow the amine captured CO₂ to be released.

### Example 2

In this example, a 12 wt. % Ni₂Mg, Cu₃Ni or Cu₂Mg catalyst impregnated on a magnesium aluminate spinel substrate was used in the CO₂C reactor to convert (CO₂) with (H₂) to a mixture of H₂ and CH₄ and CO₂ at temperatures from 275 to 350° C. Table 2 shows that the CO₂ conversion efficiency was about 86% with a greater than 99% CH₄ production selectivity.

The H₂ and CO₂ was removed with a 95% efficiency from the product stream to produce a CH₄ stream with greater than 98% purity. The minor components in the CH₄ stream include CO₂ at about 1.5 vol. %, H₂ at about 0.2%, and H₂O at about 0.3%.

The resulting CH₄ stream met pipeline quality natural gas standards.

### REFERENCES

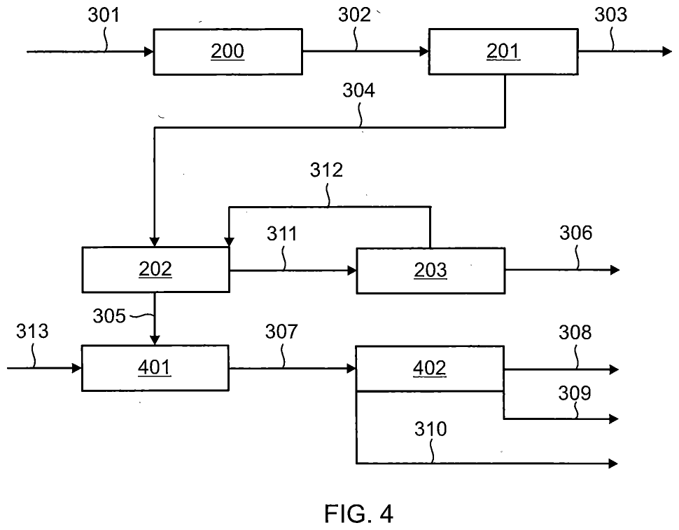

Abe, T.; Tanizawa, M.; Watanabe, K.; Taguchi, A.: CO₂ methanation property of Ru nanoparticle-loaded TiO₂ prepared by a polygonal barrel-sputtering method, Energy Environ. Sci., 2, 315-321 (2009). http://doi.org/10.1039/b817740f.
Alarcón, A.; Guilera, J.; Díaz-López, J. A.; Andreu, T.: Optimization of nickel and ceria catalyst content for synthetic natural gas production through CO₂ methanation, Fuel Process. Technol., 193, 114-122 (2019).
American Gas Association (AGA), Report No. 4A, May 2001. FIG. 2-4 Example Pipeline Quality Natural Gas.
Aryal, N., Odde, M., Petersen, C., Ditley, L., Ottosen, Vedel, M., Kofoed, W.: Methane production from syngas using a trickle-bed reactor, Bioresource Technology, 333 (2021).
Bando, K.; Soga, K.; Kunimori, K.; Ichikuni, N.; Okabe, K.; Kusama, H.; Sayama, K.; Arakawa, H.: CO₂ hydrogenation activity and surface structure of zeolite-supported Rh catalysts. Applied Catalysis A Gen., 173, 47-60 (1998). http://doi.org/10.1016/S0926-860X(98)00143-4.
Chai, S.; Men, Y.; Wang, J.; Liu, S.; Song, Q.; An, W.; Kolb, G.: Boosting CO₂ methanation activity on Ru/TiO₂ catalysts by exposing facets of anatase TiO₂. Journal of CO₂ Utilization, 33, 242-252 (2019). http://doi.org/10.1016/j.jcou.2019.05.031.
Jia, X.; Zhang, X.; Rui, N.; Hu, X.; Liu, C.-J. Structural effect of Ni/ZrO₂ catalyst on CO₂ methanation with enhanced activity, Applied Catalysis B: Environmental, 244, 159-169 (2018).
Jiang, Y.; Huang, T.; Dong, L.; Su, T.; Li, B.; Luo, X.; Xie, X.; Qin, Z.; Xu, C.; Ji, H.: Mn modified Ni/Bentonite for CO₂ methanation, Catalysts, 8, 646 (2018).
Jiang, Y.; Huang, T.; Dong, L.; Qin, Z.; Ji, H.: Ni/bentonite catalysts prepared by solution combustion method for CO₂ methanation, Chinese Journal Chem. Eng., 26, 2361-2367 (2018).
Li, Y., Chen, C., Ye, M., Imbault, A. L.: Methanation of synthesis gas to produce methane, J. Chem. Educ., 99, 1448-1453 (2022).
Li, L.; Zeng, W.; Song, M.; Wu, X.; Li, G.; Hu, C.: Research, progress, and reaction mechanism of CO₂ methanation over Ni-based catalysts at low temperature, a review, Catalysts, 12, 244 (2022).
Lin, J.; Ma, C.; Wang, Q.; Xu, Y.; Ma, G.; Wang, J.; Wang, H.; Dong, C.; Zhang, C.; Ding, M.: Enhanced low-temperature performance of CO₂ methanation over mesoporous Ni/Al₂O₃—ZrO₂ catalysts, Applied Catalysis B Environmental, 243, 262-272 (2018).
Lin, X.; Wang, S.; Tu, W.; Hu, Z.; Ding, Z.; Hou, Y.; Xu, R.; Dai, W.: MOF-derived hierarchical hollow spheres composed of carbon-confined Ni nanoparticles for efficient CO₂ methanation, Catalysis Science Technol., 9, 731-738 (2019) http://doi.org/10.1039/C8CY02329H.
Lippi, R.; Howard, S. C.; Barron, H.; Easton, C. D.; Madsen, I. C.; Waddington, L. J.; Vogt, C.; Hill, M. R.; Sumby, C. J.; Doonan, C. J.: Highly active catalyst for CO₂ methanation derived from a metal organic framework template. J. Mater. Chem. A, 5, 12990-12997 (2017). http://doi.org/10.1039/C7TA00958E.
Martin, N. M.; Hemmingsson, F.; Schaefer, A.; Ek, M.; Merte, L. R.; Hejral, U.; Gustafson, J.; Skoglundh, M.; Dippel, A.-C.; Gutowski, O.: Structure-function relationship for CO₂ methanation over ceria supported Rh and Ni catalysts under atmospheric pressure conditions, Catalysis Science Technol, 9, 1644-1653 (2019) http://doi.org/10.1039/C8CY02097C.
Nam, H.; Kim, J. H.; Kim, H.; Kim, M. J.; Jeon, S.-G.; Jin, G.-T.; Won, Y.; Hwang, B. W.; Lee, S.-Y.; Baek, J.-I.: CO₂ methanation in a bench-scale bubbling fluidized bed reactor using Ni-based catalyst and its exothermic heat transfer analysis, Energy, 214 (2020). http://doi.org/10.1016/j.energy.2020.118895.
National Academy of Sciences, Chemical Utilization of CO₂ into Chemicals and Fuels, Gaseous Carbon Waste Streams Utilization: Status and Research Needs, National Academies Press, Washington D.C. (2019).
Quindimil, A.; De-La-Torre, U.; Pereda-Ayo, B.; Davó-Quiñonero, A.; Bailón-Garcia, E.; Lozano-Castelló, D.; González-Marcos, J. A.; Bueno-López, A.; González-Velasco, J. R.: Effect of metal loading on the CO₂ methanation: A comparison between alumina supported Ni and Ru catalysts, Catalysis Today, 356, 419-432 (2020).
Romano, L.; Ruggeri, F.: Methane from syngas—Amec Foster Wheeler VESTA technology development, 81, 249-254 (2015).
Rui, N.: Highly active Ni/CeO₂ catalyst for CO₂ methanation: Preparation and characterization, Brookhaven National Laboratory Report #BNL-220907-2021-JAAM (2021).
Sabatier, P.; Senderens, J.: Direct hydrogenation of oxides of carbon in presence of various finely divided metals, C. R. Acad. Sci., 134, 689-691 (1902).
Steynberg, A. P.; Dry, M. E.; Davis, B. H.; Breman, B. B.: Fischer-Tropsch reactors, Studies in Surface Science and Catalysis, 152, 64-195 (2004).
Tada, S.; Ochieng, O. J.; Kikuchi, R.; Haneda, T.; Kameyama, H: Promotion of CO₂ methanation activity and CH₄ selectivity at low temperatures over Ru/CeO₂/Al₂O₃ catalysts, Int. J. Hydrogen Energy, 39, 10090-10100 (2014).
Tan, C. H.; Nomanbhay, S; Shamsuddin, A. H.; Park, Y-K; Hernández-Cocoletzi, H.; Show, P. L.: Current developments in catalytic methanation of CO2—a review, Frontiers in Energy Research, 9, 1-7 (2022).
Ye, R.-P.; Liao, L.; Reina, T. R.; Liu, J.; Chevella, D.; Jin, Y.; Fan, M.; Liu, J.: Engineering Ni/SiO₂ catalysts for enhanced CO₂ methanation, Fuel, 285, 119-151 (2020)
Ye, R.-P.; Li, Q.; Gong, W.; Wang, T.; Razink, J. J.; Lin, L.; Qin, Y.-Y.; Zhou, Z.; Adidharma, H.; Tang, J.: High-performance of nanostructured Ni/CeO₂ catalyst on CO₂ methanation, Applied Catalysis B Environ, 268, (2019).
Yugo, M., Soler, A: A look into the role of e-fuels in the transport system in Europe, Conservation of Clean Air and Water in Europe (CONCAWE), 28, 1-22 (2019).
Wikipedia, Fischer-Tropsch processes, en.wikipedia.org (Jun. 14, 2022).
Wikipedia, Natural gas, en.wikipedia.org (Jun. 14, 2022).
Wikipedia, Power-to-X, en.wikipedia.org (Jun. 14, 2022).

1. A process for the production of low-carbon CH₄ comprising:
a. mixing a first feed stream comprising CO₂ with a second feed stream comprising renewable H₂ to produce a CO₂ conversion reactor stream, wherein the CO₂ conversion reactor comprises a CO₂ hydrogenation catalyst, and wherein the hydrogenation catalyst is a solid solution catalyst comprising Ni and Mg or a solid solution catalyst comprising Cu and Mg;
b. feeding the CO₂ conversion reactor stream to a CO₂ conversion reactor to produce a reactor product stream comprising CH₄, CO₂, H₂ and H₂O;
c. removing the CO₂, H₂ and H₂O from the reactor product stream to produce a CH₄ stream having a purity that meets pipeline transmission specifications.

2. The process of claim 1, wherein the H₂ in the CO₂ conversion reactor stream is produced from electrolysis of H₂O using renewable electric power.

3. The process of claim 1, wherein the CO₂ in the CO₂ in the conversion reactor stream is captured from stationary sources or ambient air.

4. The process of claim 1, wherein the H₂/CO₂ ratio in in the conversion reactor stream is at a 4.0/1.0 (v/v) ratio for the production of CH₄.

5. The process of claim 1, wherein the CO₂ conversion reactor is a multi-tubular, fixed bed reactor.

6. The process of claim 1, wherein the hydrogenation catalyst comprises Ni₂Mg, Cu₃Ni or CuMg.

7. The process of claim 6, wherein the hydrogenation catalyst is in the form of a pellet or a tablet.

8. The process of claim 6, wherein the hydrogenation catalyst further comprises one or more spinels at a concentration between 0.01 wt. % and 25 wt. %.

9. The process of claim 8, wherein the one or more spinels are selected from a group consisting of magnesium aluminate, zinc aluminate and nickel aluminate, and wherein the surface area of the one or more spinels ranges from 10 m²/g to 100 m²/g.

10. The process of claim 6, wherein the hydrogenation catalyst is chemically and physically stable between 0° C. and 1,100° C.

11. The process of claim 6, wherein the hydrogenation catalyst is activated, and wherein it is activated in-situ by reduction with H2 between 275° C. and 350° C.

12. The process of claim 6, wherein the percentage conversion of CO₂ to CH₄ is between 85% and 99.9% between 275° C. and 350° C.

13. The process of claim 6, wherein the ratio of CH₄ to CO₂, H₂ and H₂O produced is between 99:1 and 99.9:1.

14. The process of claim 6, wherein the CO₂ conversion reactor is cooled by the production of steam.

15. The process of claim 6, wherein the hydrogenation catalyst is cooled by a high-temperature heat transfer fluid.

16. The process of claim 4, wherein the CO₂ conversion reactor stream is diluted with additional H₂ in excess of the stoichiometric 4.0/1.0 H₂/CO₂ ratio to assist in cooling of the catalyst.

17. The process of claim 1, wherein the CO₂ reactor product stream is fed to a product processing unit comprising CO₂, H₂O and H₂ removal systems.

18. The process of claim 17, wherein CH₄ stream meets natural gas pipeline specifications.

19. A hydrogenation catalyst, wherein the hydrogenation catalyst is a solid solution catalyst comprising Ni and Mg or a solid solution catalyst comprising Cu and Mg.

20. The hydrogenation catalyst of claim 19, wherein the hydrogenation catalyst further comprises one or more spinels at a concentration between 0.01 wt. % and 25 wt. %.

21. The hydrogenation catalyst of claim 20, wherein the one or more spinels are selected from a group consisting of magnesium aluminate, zinc aluminate and nickel aluminate, and wherein the surface area of the one or more spinels ranges from 10 m²/g to 100 m²/g.

22. The hydrogenation catalyst of claim 21, wherein the hydrogenation catalyst is chemically and physically stable between 0° C. and 1,100° C.

23. The hydrogenation catalyst of claim 22, wherein the hydrogenation catalyst is in the form of a pellet or a tablet.

24. The hydrogenation catalyst of claim 23, wherein the hydrogenation catalyst comprises Ni₂Mg, Cu₃Ni or CuMg.
